(19) United States
(12) Patent Application Publication
(10) Pub. No.: US 2014/0080794 A1
(43) Pub. Date: Mar. 20, 2014

(54) Methods and Compositions for the Treatment of Estrogen-Dependent Hyperproliferative Uterine Disorders

(71) Applicant: Vivus, Inc., Mountain View, CA (US)

(72) Inventors: Changjin Wang, San Diego, CA (US); Leland F. Wilson, Menlo Park, CA (US)

(21) Appl. No.: 13/945,430

(22) Filed: Jul. 18, 2013

Related U.S. Application Data
(63) Continuation of application No. 12/587,004, filed on Sep. 29, 2009, now abandoned.
(60) Provisional application No. 61/194,491, filed on Sep. 29, 2008.

(51) Int. Cl. A61K 31/565; A61K 31/192; A61K 31/196; A61K 31/5415; A61K 31/4535; A61K 31/4196; A61K 31/4025

(52) U.S. Cl. CPC A61K 31/565; A61K 31/4196; A61K 31/192; A61K 31/4025; A61K 31/5415; A61K 31/4535; A61K 31/196
USPC 514/171; 514/383; 514/226.5

(57) ABSTRACT

The present invention relates to the treatment of estrogen-dependent hyperproliferative uterine disorders including endometriosis, uterine fibroids, endometrial hyperplasia, uterine cancer, and their related symptoms by intravaginally administering at least two active agents selected from an aromatase inhibitor, an antiinflammatory agent, and a uterine-selective estrogen receptor antagonist. This combination therapy reduces local estrogen production, blocks local estrogen action, and suppresses inflammation locally, resulting in starvation of the estrogen-dependent diseased tissues, relief of related symptoms, and retardation of disease progression. Intravaginal delivery maximizes local inhibition of estrogen production without significantly affecting systemic circulating estrogen levels. This results in enhanced clinical efficacy and reduced side effects.

### CROSS-REFERENCE TO RELATED APPLICATIONS

This application is a continuation of U.S. patent application Ser. No. 12/587,004, filed Sep. 29, 2009, which claims priority under 35 U.S.C. §119(e) to Provisional U.S. Patent Application Ser. No. 61/194,491, filed Sep. 29, 2008, the disclosure of each is incorporated by reference herein.

### FIELD OF THE INVENTION

The invention relates generally to pharmaceutical methods and compositions in which two or more active agents, as will be specified, are combined for the treatment of estrogen-dependent hyperproliferative uterine disorders such as endometriosis, uterine fibroids, endometrial hyperplasia, uterine cancer, and related conditions. As such, the invention finds utility in the fields of medicine, pharmaceutical formulation, and drug delivery.

### BACKGROUND

Endometriosis is an estrogen-dependent disease that affects about four to six million American women of reproductive age and is the most common cause of chronic pelvic pain (Giudice et al. (2004) Lancet 1364:1789-99). Endometriosis is characterized by the presence of endometrium-like tissue, including endometrial glandular and stromal cells, outside the uterus, primarily on pelvic peritoneum and ovaries, bowel, rectum, and bladder. Endometriosis is largely a condition seen in pre-menopausal women. Symptoms of endometriosis include pelvic pain, dysmenorrhorea and dyspareunia, and are often found to be associated with infertility (i.e., the inability to conceive) as well as “sub-fertility” (i.e., diminished reproductive capacity). Endometriosis is now believed to be caused by genetic factors, and there are a number of theories proposed for the origin and processes involved in the pathogenesis of the disease. Nevertheless, it is now recognized that the growth of endometrial tissue depends on the presence of estrogen, and therapeutic agents that reduce estrogen levels have been administered with some relief. The only long-term solution to endometriosis, however, is still surgical intervention. While surgery may be effective, recurrence of endometriosis within five years is not uncommon, and post-surgical adhesions can be at least as problematic as the disease itself.

Uterine fibroids, also known as myomas (short for leiomyomas; also termed fibromas and myofibromas) are the most common benign tumors of the female reproductive tract. Uterine fibroids are believed to be associated with exposure to elevated levels of estrogen. They occur in or on the surface of the uterus; specifically, they may be subserous, present on the exterior surface of the uterus; intracavitary, lying within the uterine cavity; intramural, within the uterine wall; or submucosal, located partially within the uterine wall and partially within the uterine cavity. The likelihood of developing uterine fibroids increases with age until menopause. Around 20% of women in their twenties and about 40% of women in their forties have fibroids. Depending on the size, location and number of fibroid tumors, symptoms can include abnormal gynecologic hemorrhaging, heavy or painful menstrual periods, metrorrhagia (i.e., vaginal bleeding between periods), cramping, abdominal discomfort or bloating, backache, urinary frequency or retention, and in some cases, infertility or subfertility. Surgical procedures such as myomectomies and hysterectomies are the most common therapeutic approaches.

Uterine cancer refers to several types of cancers that occur in the uterus, including endometrial cancer, uterine sarcomas, and cervical cancer. These cancers are sometimes preceded by a condition known as endometrial hyperplasia, in which there is a measurable increase in the number of endometrial cells lining the uterus. Endometrial cancer is the most common gynecologic cancer in the United States, with most incidences occurring in premenopausal and perimenopausal women who are exposed to an excess of estrogen and in postmenopausal women with some degree of endometrial atrophy. Uterine tumors have been found to have high levels of estrogen receptors, and their growth has been found to be enhanced by estrogen, particularly unopposed estrogen exposure (i.e., estrogen administered in the absence of a progestagen such as progesterone). Endometrial cancer is the third most common cause of gynecologic cancer death (behind ovarian and cervical cancer). A total abdominal hysterectomy with bilateral salpingo oophorectomy (surgical removal of both the Fallopian tubes and ovaries) is the most common therapeutic approach.

Studies have shown that hyperproliferative uterine disorders such as endometriosis, uterine fibroids, endometrial hyperplasia, and uterine cancers are highly responsive to estrogen for growth, as noted above. Drug therapy intended to inhibit estrogen production at the ovaries has been suggested and is in some instances used. For instance, GnRH (gonatrophic hormone releasing hormone) agonists and antagonists have been used to shut down estrogen production and lower circulating estrogen concentration. Although this approach has resulted in some reduction in pelvic pain and regression of fibroids, the resulting hypo-estrogenic effect can lead to undesirable and potentially serious side effects, including hot flashes, vaginal bleeding and dryness, decreased libido, breast tenderness, insomnia, depression, decreased elasticity of the skin, and osteoporosis. Thus, administration of these agents is generally limited to a six-month period, and the incidence of recurrence of the pre-treatment symptoms is high.

Recent studies have demonstrated high aromatase expression and activity in endometriotic tissues and fibroids. Thus, aromatase inhibitors have been suggested for the treatment of endometriosis and fibroids. Amsterdam et al. (2005) Fertil. Steril. 84 (2): 300-304, for instance, reported symptom improvement in endometriosis patients taking 1 mg anastrozole and an oral contraceptive formulation containing 20 μg ethinyl estradiol/1 mg levonorgestrel per day for six months. The estradiol-containing oral contraceptive was used to replenish systemic estradiol depleted by the aromatase inhibitor. Systemic estradiol levels nevertheless remained significantly suppressed in all patients by the end of the treatment period.

Published U.S. Patent Application No. 2005/0049231 A1 to Knox et al. suggests a method for the treatment of endometriosis and uterine fibroids by intravaginally administering an aromatase inhibitor (such as, for example, anastrozole, exemestane or letrozole). The method is intended to reduce the systemic effect on estrogen and minimize various adverse side effects when compared with the oral delivery of aromatase inhibitors. Aromatase is an enzyme that catalyzes a reaction to form estrogen. As suggested by recent research, local inflammation and production of prostaglandins in endometriotic tissues and uterine fibroids promote aromatase overexpression, which in turn increases local biosynthesis of estrogen. A localized elevated level of estrogen not only feeds the growth of the disease tissues (via estrogen receptors in the disease tissues) but also stimulates local inflammation and prostaglandin production. Hence there is a feedback loop that fuels local estrogen production and disease progression. An aromatase inhibitor delivered intravaginally (as proposed by Knox et al.) would inhibit aromatase activity and decrease estrogen production locally, but do little if anything to disrupt the feedback loop or counter the unwanted side effects, primarily local inflammation and prostaglandin production.

As may be concluded from the foregoing, there is an ongoing need in the art for a non-surgical methodology for the treatment of hyperproliferative uterine disorders wherein local estrogen levels are reduced pharmacologically without the adverse side effects associated with aromatase inhibitor monotherapy as described in Knox et al.

It is, therefore, an object of the present invention to provide methods and compositions for the treatment of hyperproliferative uterine disorders such as endometriosis, uterine fibroids, endometrial hyperplasia, uterine cancer, and related conditions that overcome the aforementioned limitations and disadvantages of the art.

### SUMMARY OF THE INVENTION

Accordingly, the present invention addresses the aforementioned need in the art by providing methods and compositions for the treatment of estrogen-dependent hyperproliferative uterine disorders.

In one embodiment, a method is provided for treating a subject for an estrogen-dependent hyperproliferative uterine condition, wherein the method comprises intravaginally administering to the subject a combination of at least two active agents selected from an aromatase inhibitor, an antiinflammatory agent, and a uterine-selective estrogen receptor antagonist. Generally, although not necessarily, one of the active agents is an aromatase inhibitor, in which case the aromatase inhibitor may be combined with the antiinflammatory agent, the uterine-selective estrogen receptor antagonist, or both. Estrogen-dependent hyperproliferative uterine conditions include, without limitation, endometriosis, uterine fibroid tumor(s), endometrial hyperplasia, and uterine cancers.

The active agents are normally administered on a regular basis throughout a dosing period, which may be on the order of hours, a day, several days, or more; typically the dosing period is at least a week, and in some cases at least a month. The agents may be administered separately, e.g., at different times of day and/or in different compositions, dosage forms, or delivery systems, or the agents may be administered simultaneously. In the latter case, the agents will typically although not necessarily be incorporated into a single composition or delivery system adapted for intravaginal drug administration. I

[symbol]

The uterine-selective estrogen receptor antagonist is selected from the subset of selective estrogen receptor modulators that exhibit estrogen antagonist activity in the uterus but may exhibit estrogen agonist behavior in other regions of the body, e.g., in the bone or cardiovascular systems. The uterine-selective estrogen receptor antagonist minimizes the action of locally present estrogens. The aromatase inhibitor reduces the overexpression of aromatase, in turn reducing the local biosynthesis of estrogens. The antiinflammatory agent mitigates local inflammation resulting from elevated levels of estrogen. The combination of two or all three of these agents is clinically efficacious at low doses of each individual agent, i.e., low relative to the dose of each individual agent that would be required in monotherapy, in turn providing a better safety profile and a reduction in side effects. Moreover, the combination does not significantly lower systemic estrogen levels.

The active agents may be administered separately or in combination; in the latter case they are normally combined in a pharmaceutical composition for intravaginal administration as will be described herein.

With respect to the various combinations of the three types of active agents:

An aromatase inhibitor in combination with an antiinflammatory agent delivered intravaginally would break the “positive feedback loop” referred to above by suppressing aromatase expression, activity, and local inflammation, resulting in significant reduction of local estrogen production and retardation of disease progression;

An aromatase inhibitor in combination with a uterine-selective estrogen receptor antagonist would have a similar effect as the aromatase inhibitor, suppressing suppress aromatase activity, while the uterine-selective estrogen receptor antagonist would antagonize estrogen effect locally to inhibit disease tissue growth;

An antiinflammatory agent in combination with a uterine-selective estrogen receptor antagonist delivered intravaginally would have a similar effect as the antiinflammatory agent by suppressing local inflammation and inhibiting aromatase overexpression, and the uterine-selective estrogen receptor antagonist would antagonize local estrogen effects to inhibit disease tissue growth; and

An aromatase inhibitor in combination with both an antiinflammatory agent and a uterine-selective estrogen receptor antagonist delivered intravaginally would have a synergistic effect as they would break the “positive feedback loop” referred to above by suppressing aromatase expression, activity, and local inflammation, resulting in significant reduction of local estrogen production, and blocking estrogen effect locally to inhibit disease tissue growth.

Thus, this combination therapy reduces estrogen production and action locally, resulting in starvation of the estrogen-dependent disease tissues, relief of disease-related symptoms, and retardation of disease progression, achieving symptom relief and disease modifying therapeutic efficacy. Intravaginal delivery maximizes local inhibition of estrogen production in the disease tissues while not significantly affecting systemic level of circulating estrogen, which have been produced by the ovaries under normal physiological hormone regulation. This results in better clinical efficacy and reduced side effects, which will allow for longer-term treatment than current therapies.

In another embodiment, compositions and delivery systems are provided for intravaginal drug administration, wherein the compositions and delivery systems contain at least two of an aromatase inhibitor, a nonsteroidal antiinflammatory drug, and a uterine-selective estrogen receptor antagonist. Typically, the compositions and delivery systems contain the aromatase inhibitor and either the nonsteroidal antiinflammatory drug, the uterine-selective estrogen receptor antagonist, or both. In one version of this embodiment, then, the pharmaceutical composition or delivery system containing the composition comprises an aromatase inhibitor, an antiinflammatory agent, preferably a nonsteroidal antiinflammatory drug, and a hyperproliferative uterine-selective estrogen antagonist, each in an amount effective for the treatment of an estrogen-dependent hyperproliferative uterine disorder when administered in combination intravaginally, and a pharmaceutical carrier suitable for incorporation into an intravaginally administrable formulation.

In a further embodiment of the invention, the pharmaceutical composition or delivery system containing the composition includes, and may consist essentially of, of an aromatase inhibitor, a nonsteroidal antiinflammatory drug, a hyperproliferative uterine-selective estrogen antagonist, each in an amount effective for the treatment of an estrogen-dependent hyperproliferative uterine disorder when administered in combination intravaginally, and a pharmaceutical carrier suitable for incorporation into an intravaginally administrable formulation.

In yet another embodiment of the invention, the pharmaceutical composition or delivery system containing the composition includes, and may consist essentially of, an aromatase inhibitor, a nonsteroidal antiinflammatory drug, each in an amount effective for the treatment of an estrogen-dependent hyperproliferative uterine disorder when administered in combination intravaginally, and a pharmaceutical carrier suitable for incorporation into an intravaginally administrable formulation.

In still another embodiment of the invention, the pharmaceutical composition or delivery system containing the composition includes, and may consist essentially of, an aromatase inhibitor, a hyperproliferative uterine-selective estrogen antagonist, each in an amount effective for the treatment of an estrogen-dependent hyperproliferative uterine disorder when administered in combination intravaginally, and a pharmaceutical carrier suitable for incorporation into an intravaginally administrable formulation.

In yet another embodiment of the invention, the pharmaceutical composition or delivery system containing the composition includes, and may consist essentially of, a nonsteroidal antiinflammatory drug, a hyperproliferative uterine-selective estrogen antagonist, each in an amount effective for the treatment of an estrogen-dependent hyperproliferative uterine disorder when administered in combination intravaginally, and a pharmaceutical carrier suitable for incorporation into an intravaginally administrable formulation.

In additional embodiments of the invention, the pharmaceutical composition or delivery system containing the composition includes, and may consist essentially of:

An aromatase inhibitor selected from anastrozole, exemestane and letrozole; a nonsteroidal anti-inflammatory drug selected from aceclofenac, aspirin, dexketoprofen, diclofenac, diflunisal, etodolac, fenoprofen, flurbiprofen, flufenamic acid, ibuprofen, indomethacin, ketoprofen, ketorolac, lornoxicam, meclofenamic acid, mefenamic acid, meloxicam, nabumetone, naproxen, oxaprozin, piroxicam, sulindac, tiaprofenic acid, and tolmetin; and a uterine-selective estrogen receptor antagonist selected from the group consisting of fulvestrant, ormeloxifene and raloxifene, with each active agent present in an amount effective for the treatment of an estrogen-dependent hyperproliferative uterine disorder when administered in combination intravaginally; and a pharmaceutically acceptable carrier suitable for incorporation into an intravaginally administrable formulation;

An aromatase inhibitor, a nonsteroidal antiinflammatory drug selected from aceclofenac, aspirin, dexketoprofen, diclofenac, diflunisal, etodolac, fenoprofen, flurbiprofen, flufenamic acid, ibuprofen, indomethacin, ketoprofen, ketorolac, lornoxicam, meclofenamic acid, mefenamic acid, meloxicam, nabumetone, naproxen, oxaprozin, piroxicam, sulindac, tiaprofenic acid, and tolmetin, with each active agent present in an amount effective for the treatment of an estrogen-dependent hyperproliferative uterine disorder when administered in combination intravaginally, a pharmaceutically acceptable carrier suitable for incorporation into an intravaginally administrable formulation;

An aromatase inhibitor, a uterine-selective estrogen receptor antagonist selected from fulvestrant, ormeloxifene and raloxifene, with each active agent present in an amount effective for the treatment of an estrogen-dependent hyperproliferative uterine disorder when administered in combination intravaginally, and a pharmaceutically acceptable carrier suitable for incorporation into an intravaginally administrable formulation;

A nonsteroidal antiinflammatory drug, a therapeutically effective amount of a uterine-selective estrogen receptor antagonist selected from fulvestrant, ormeloxifene and raloxifene, with each active agent present in an amount effective for the treatment of an estrogen-dependent hyperproliferative uterine disorder when administered in combination intravaginally, and a pharmaceutically acceptable carrier suitable for incorporation into an intravaginally administrable formulation;

An aromatase inhibitor, a nonsteroidal antiinflammatory drug, a uterine-selective estrogen receptor antagonist, with each active agent present in an amount effective for the treatment of an estrogen-dependent hyperproliferative uterine disorder when administered in combination intravaginally, and a pharmaceutically acceptable carrier suitable for incorporation into an intravaginally administrable formulation.

### DETAILED DESCRIPTION OF THE INVENTION

It is to be understood that unless otherwise indicated this invention is not limited to particular embodiments described, as such may vary. It is also to be understood that the terminology used herein is for the purpose of describing particular embodiments only, and is not intended to be limiting. Unless defined otherwise, all technical and scientific terms used herein have the same meaning as commonly understood by one skilled in the art to which this invention belongs.

As used herein and in the appended claims, the singular forms “a,” “and,” and “the” include plural referents unless the context clearly dictates otherwise. Thus, for example, “an aromatase inhibitor” encompasses a plurality of aromatase inhibitors as well as a single aromatase inhibitor, reference to “a pharmaceutically acceptable carrier” is intended to encompass a plurality of pharmaceutically acceptable carriers as well as a single such carrier, and so forth.

In this specification and in the claims that follow, reference will be made to a number of terms, which shall be defined to have the following meanings:

“Optional” or “optionally present”—as in an “optionally present” additive or an “optionally present” active agent—means that the subsequently described component (e.g., additive or active agent) may or may not be present, so that the description includes instances where the component is present and instances where it is not.

By “pharmaceutically acceptable” is meant a compound that is not biologically or otherwise undesirable, e.g., the compound may included in a pharmaceutical composition that is administered to a subject as described herein without causing any significant undesirable biological effects or interacting in a deleterious manner with any of the other components of the composition. When the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition.

The term “active agent” refers to any chemical compound, complex or composition that exhibits a desirable effect in the biological context, i.e., when administered to a subject or evaluated in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “active agent” is used, or when a particular active agent is specifically identified, then, it is to be understood that pharmaceutically acceptable salts, esters, amides, prodrugs, active metabolites, isomers, analogs, etc. of the agent are intended as well as the agent per se.

By an “effective” amount or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

“Carriers” or “excipients” as used herein refer to conventional pharmaceutically acceptable carrier and excipient materials suitable for intravaginal drug administration, and include any such materials known in the art that are nontoxic and do not interact with other components of a pharmaceutical composition or drug delivery system in a deleterious manner.

The term “about” as used herein may be applied to modify any quantitative representation that could permissively vary without resulting in a change in the basic function to which it is related.

“Aromatase inhibitor” refers to a chemical compound that blocks or inhibits the activity of aromatase which is an enzyme that converts androgens to estrogens. As such, an aromatase inhibitor acts to reduce estrogen levels in the body.

A “side effect” refers to a consequence other than the purpose or indication for which an active agent is administered, i.e., adverse effects produced by an active agent, particularly on a tissue or organ system other than the one intended to benefit from administration of the active agent. The invention herein reduces the side effects associated with administration of aromatase inhibitors alone, wherein “reducing the side effects” is intended to encompass the prevention, elimination, or diminution of one or more side effects.

The term “subject” refers to a mammalian being, generally a female human being.

The term “controlled release” is intended to refer to any pharmaceutical formulation in which release of the drug is not immediate, i.e., with a “controlled release” formulation, intravaginal administration does not result in immediate release of the all of the active agent. The term is used interchangeably with “nonimmediate release” as defined in Remington: The Science and Practice of Pharmacy, 20^{th} edition (Lippincott Williams & Wilkins, 2000).

The term “sustained release” is used in its conventional sense to refer to a drug formulation that provides for gradual release of a drug over an extended period of time, and that preferably, although not necessarily, results in substantially constant blood levels of a drug over an extended time period. A sustained release formulation may be administered once to provide a single bolus dose of the drug, which is then effective for up to a day or even up to several days.

“Intravaginal” administration refers to a mode of drug administration wherein the active agents herein are administered via the vagina so that the agents are locally absorbed by the vaginal mucosa. Intravaginal administration provides for rapid delivery of the agents to localized areas and tissues such that therapeutically effective drug concentrations are achieved locally, in the region of the diseased or otherwise abnormal tissue, i.e., the tissues or organs in proximity to the vagina, such as the uterus. Although significant local levels of the active agents are provided, circulating blood levels, i.e., systemic levels, are sufficiently low to avoid or minimize the side effects typically encountered with system, e.g., oral, drug administration.

By “estrogen-dependent hyperproliferative uterine disorder” is meant a hyperproliferative uterine condition or disease that is estrogen-induced and/or estrogen stimulated; in the latter case, the common pathology involves exacerbation of the condition or disease by elevated estrogen levels. A “hyperproliferative” disorder is one wherein proliferation of cells exceeds that normally seen, and wherein the hyperproliferation involves cells and tissues within or in the region of the uterus.

The invention, in one embodiment, involves treatment of a female subject afflicted with an estrogen-dependent hyperproliferative uterine disorder with a combination of at least two active agents selected from an aromatase inhibitor, an antiinflammatory agent, particularly a nonsteroidal antiinflammatory drug, and a uterine-selective estrogen receptor antagonist.

The aromatase inhibitor can be steroidal or nonsteroidal. Steroidal aromatase inhibitors include, but are not limited to: substituted androst-4-ene-3,17-diones and analogs thereof, including androst-4-ene-3-ones, androst-4-ene-3,6,17-triones, androsta-1,4-diene-3,17-diones, androsta-1,4,6-triene-3,17-diones, and androsta-1,4,6-triene-3,6,17-triones; norethindrone analogs such as 5a-dihydronorethindrone; and substituted estrenes and estrene analogs, including 4-estrene-3,17-diones, 4-estrene-3-ones, 4-estra-1,4-dien-3-ones, 4-estra-1,4-diene-3,17-diones, 4-estra-4,9(11)-diene-3,17-diones, and estra-4,9-diene-3-ones (such as mifepristone or RU486). Representative examples of steroidal aromatase inhibitors are as follows:

6-methyleneandrosta-1,4-diene-3,17-dione (exemestane);

4-hydroxyandrost-4-ene-3,17-dione (formestane);

4-aminoandrosta-1,4,6-triene-3,17-dione (minemestane);

1-methylandrosta-4,6-diene-3,17-dione (atamestan);

D-homo-17α-oxaandrosta-1,4-diene-3,17-dione (testolactone);

androsta-1,4,6-triene-3,17-dione;

2,2-dimethyl-4-hydroxyandrost-4-ene-3,17-dione;

3-hydroxyandrost-4-ene-6,17-dione;

3α-methoxyandrost-4-ene-6,17-dione;

4-(phenylthio)androst-4-ene-3,17,dione;

4-acetoxyandrost-4-ene-3,17-dione;

4-bromoacetoxy-4-androst-4-ene-3-one;

4-am inoandrost-4-ene-3,17-dione;

4-methoxyandrost-4-ene-3,17-dione;

6-hydroperoxyandrost-4-ene-3,17-dione;

6-hydroiminoandrost-4-ene-3,17-dione;

6-methyleneandrosta-1,4-diene-3,17-dione;

7-(4′-amino)phenylthioandrost-4-ene-3,17-dione;

7-((4′-aminophenyl)thio)-androsta-1,4-diene-3,17-dione;

7-phenylandrosta-1,4,6-triene-3,17-dione;

7-benzylandrosta-1,4,6-triene-3,17-dione;

14-hydroxyandrost-4-ene-3,6,17-trione;

17-bromoacetoxyandrost-4-ene-3-one;

19-mercaptoandrost-4-ene-3,17-dione;

19-aminoandrost-4-ene-3,17-dione;

19,19-difluoroandrost-4-ene-17-one;

19-ethynyl-19-hydroxyandrost-4-ene-17-one;

19-cyclopropylaminoandrost-4-ene-17-one;

2,19-(methyleneoxy)androst-4-ene-3,17-dione;

19-azidoandrost-4-ene-3,17-dione;

19-thiomethylandrost-4-ene-3,17-dione;

19-(ethyldithio)androst-4-ene-3,17-dione;

19-thioandrost-4-ene-3,17-dione;

19-chloroandrost-4-ene-3,17-dione;

norethindrone;

5α-dihydronorethindrone;

norethisterone;

10-fluoroethindrone;

4-estrene-3,17-dione;

4-estrene-3,6,17-d ione;

10-thiirane-4-estrene-3,17-dione;

10-oxirane-4-estrene-3,17-dione;

10-amino-4-estrene-3,17-dione;

10-hydroperoxy-4-estrene-3,17-dione;

10-(2-propynyl)-4-estrene-3,17-dione;

10-propargyl-4-estrene-3,17-dione;

10-methylthioethylestra-4,9(11)-diene-3,17-dione;

17β-hydroxy-10-mercapto-4-estrene-3-one;

17β-hydroxy-10-methylthioestra-1,4-dien-3-one; and

mifepristone (RU486).

Nonsteroidal aromatase inhibitors useful in conjunction with the method of the invention include, for example: aminoglutethimides, azoles, including imidazoles, triazoles, and tetrazoles; other nitrogen heterocycles such as pyrimidines, pyridines, piperidines, piperidones, pyrrolidines, pyrrolidinones, oxazines, and indolizinones; flavonoids; hydroxybenzoic acids; nicotine derivatives; chlorobenzenes; pantetheines; and ergosterol and ergosterol derivatives. Representative examples of such nonsteroidal aromatase inhibitors include the following:

Aminoglutethimides:
pyridoglutethimide (rogletimid);
aminoglutethimide;
N-octylpyridoglutethimide;
C-octylpyridoglutethimide;

Azoles:
α, α, α′, α′-tetramethyl-5-(1H-1,2,4-triazol-1-ylmethyl)-1,3-benzene-diacetonitrile (anastrozole);
4,4′-(1H-1,2,4-triazol-1-ylmethylene)bisbenzonitrile (letrozole);
6-[(S)-4-chlorophenyl)-1H-1,2,4-triazol-1-ylmethyl)]-1-methyl-1H-benzotriazole (vorozole);
4-(5,6,7,8-tetrahydroimidazo[1,5-a]pyridin-5-yl)benzonitrile (fadrozole);
4-[N-bromobenzyl]-N-(4H-1,2,4-triazol-4-yl)amino) benzonitrile

(YM 511);
2-(imidazol-1-yl)-4,6-dimorpholino-1,3,5-triazine;
4-(5,6,7,8-tetrahydroimidazo[1,5-a]pyridin-5-yl)benzonitrile hydrochloride (CGS 16949A);
4,4′-(2H-tetrazol-2-ylmethylene)benzonitrile (CGP 47645);
bis(p-cyanophenyl)imidazo-1-yl methane hemisuccinate (CGS 18320B);
cis-1-[(4-(1-imidazolylmethyl)cyclohexyl)methyl]imidazole succinic acid

(CGS 14796C);
1-[(benzofuran-2-yl)(4′-cyanophenyl)methyl]-1H-1,2,4-triazole (MEN 11066);
2-N,N-dimethylamino-4,6-bis(1-1-1-imidazol-1-yl)-1,3,5-triazine;

4,4′-(fluoro-1H-1,2,4-triazol-1-ylmethylene)benzonitrile (CGP 45688);
finrozole (MPV 2231 ad);

Other nonsteroidal nitrogen heterocycle-containing aromatase inhibitors:
5-(bis(4-chlorophenyl)methyl)pyrimidine (LY 56110);
1-(4-hydroxy-1-oxoundecyl)-2-(3-pyridinyl)-3-piperidine;
3-(4′-aminophenyl)pyrrolidine-2,5-dione;
3-(4′-aminophenyl)pyrrolidine-2,5-dione;
3-(cyclohexylmethyl)-1-(4-aminophenyl)-3-azabicyclo(3.1.0)hexane-2,4-dione;
3-(4-aminophenyl)-3-cyclohexylpiperidine-2,6-dione
N-(4-hydroxyundecanoyl)anabasine;
3-butyl-3-(4-pyridyl)piperidine-2,6-dione;
1-pentyl-3-(4-aminophenyl)pyrrolidine-2,5-dione;
4-tert-butylcyclohexyl-4-pyridylacetate;
MR 20492 and MR 20494 (indolizinone derivatives);
3-aminophenoxazone;
N-substituted 1,2-oxazines (Tinant et al. (1991) Acta Cryst. C47:827-9);
other aromatase inhibitors described in Karjalainen et al. (2000), European Journal of Pharmaceutical Sciences 11 (2):109-131;

Flavonoids:
flavone;
α-naphthoflavone;
chrysin;

Hydroxybenzoic acids:
4-(2,6-dihydroxybenzoyl)-3-formyl-5-hydroxybenzoic acid;

Nicotine, nicotine analogs, and metabolites:
nicotine;
N-octanoylnornicotine;
cotinine;

Chlorobenzenes:
LY 183648;

Pantetheine compounds:
FR 901537;

Ergosterol compounds:
ergosterol;
ergosterol-5,8-peroxide;
5α,8α-epidioxy-(22E,24R)-ergosta-6,22-dien-3β-ol;
5α,8α-epidioxy-(24S)-ergosta-6,22-dien-3β-ol; and
(22E,24R)-ergosta-7,22-dien-3β,5α,6β-triol (cerevisterol).

Generally, although not necessarily, the preferred aromatase inhibitor for use in conjunction with the present method and compositions are anastrozole, exemestane, and letrozole.

The nonsteroidal antiinflammatory drugs (NSAIDS) that can be used in conjunction with the present method and compositions include, without limitation:

salicylic acid and salicylic acid derivatives such as salicylic acid per se, acetylsalicylic acid (aspirin), methyl salicylate, aloxiprin, diflunisal, salsalate, olsalazine, and sulfasalazine;

p-aminophenol derivatives such as acetaminophen;

acetic acid derivatives such as indomethacin, sulindac, etodolac, tolmetin, zomepirac, diclofenac, alclofenac, bumadizone, lonazolac, fentiazac, acemtacin, difenpiramide, oxymetacin, proglumetacin, ketorolac, aceclofenac, and bufexamac;

fenamates (derivatives of N-phenylanthranilic acid) and analogs thereof, such as mefanamic acid, flufenamic acid, meclofenamic acid, tolfenamic acid, and meclofenamate. sodium;

propionic acids such as ibuprofen, dexibuprofen, naproxen, fenoprofen, ketoprofen, dexketoprofen, benoxaprofen, indoprofen, pirprofen, carprofen, pranoprofen, alminoprofen, flunoxaprofen, vedaprofen, butibufen, fenbufen, suprofen, indoprofen, ibupiroxam, oxaprozin, flurbiprofen, tepoxalin, and tiaprofenic acid;

enolic acids, i.e., “oxicams” such as piroxicam, meloxicam, lornoxicam, cinnoxicam, droxicam, sudoxicam, and tenoxicam;

pyrazolidine derivatives and analogs such as phenylbutazone, apazone, oxyphenbutazone, mofebutazone, clofezone, kebuzone, feprazone, suxibuzone, antipyrine, aminopyrine, and dipyrone;

selective COX-2 inhibitors, including diaryl-substituted furanones, diarylsubstituted pyrazoles, indole acetic acids, and sulfonanilides, such as rofecoxib, celecoxib, parecoxib, valdecoxib, etoricoxib, lumiracoxib, firocoxib, robenacoxib, mavacoxib, and cimicoxib; and

other NSAIDs including nabumetone, niflumic acid, glucosamine, benzydamine, glucosaminoglycan polysulfate, proquazone, orgotein, nimesulide, diacerein, morniflumate, tenidap, and oxaceprol.

Preferred NSAIDs for use in conjunction with the methods and compositions of the invention include, without limitation, aceclofenac, aspirin, dexketoprofen, diclofenac, diflunisal, etodolac, fenoprofen, flurbiprofen, flufenamic acid, ibuprofen, indomethacin, ketoprofen, ketorolac, lornoxicam, meclofenamic acid, mefenamic acid, meloxicam, nabumetone, naproxen, oxaprozin, piroxicam, sulindac, tiaprofenic acid, and tolmetin, and particularly preferred NSAIDs for use herein include, for example, aspirin, diclofenac, ibuprofen, indomethacin, lornoxicam, meloxicam, naproxen and piroxicam.

The uterine-selective estrogen receptor antagonist is a selective estrogen receptor modulator, or “SERM,” that exhibits an estrogen antagonist effect in the uterus. While many patents and publications in the last ten to fifteen years have described SERMs that exhibit estrogen antagonist activity in the breast and estrogen agonist activity elsewhere in the body, e.g., in bone and in the cardiovascular system, very few SERMs have been found to have an estrogen antagonist effect in the uterus. Such compounds would be ideal in treating estrogen-dependent hyperproliferative disorders of the uterus, such as endometriosis, uterine fibroids, and the like. SERMs that act as estrogen antagonists in the uterus, i.e., the uterine-selective estrogen receptor agonists useful herein, include, without limitation: raloxifene; 2,3-dihydroraloxifene; ormeloxifene; lasofoxifene; arzoxifene; femarelle; fulvestrant; 6-(4-methanesulfonylphenyl)-5-[4-(2-piperidin-1-yl-ethoxy)phenoxy]naphthalen-2-ol (LY 2066948), and other such compounds described in Published U.S. Patent Application No. 2007/0111988 A1 to Daily et al.; and (+)-7,9-difluoro-5-[4-(2-piperidin-1-ylethoxy)phenyl]-5H-6-oxachrysen-2-ol (LSN-2120310) and other such compounds described in Published U.S. Patent Application No. 2009/0023917 A1 to Daily et al. Preferred uterine-selective estrogen receptor antagonists for use in conjunction with the present method and compositions include raloxifene, ormeloxifene, and raloxifene.

Any of the active agents may be administered, if desired, in the form of a salt, ester, amide, prodrug, conjugate, active metabolite, isomer, analog, crystalline form, hydrate, or the like, provided that the salt, ester, amide, prodrug, active metabolite, isomer, analog, crystalline form, hydrate, etc is pharmaceutically acceptable and pharmacologically active in the present context. Salts, esters, amides, prodrugs, conjugates, etc. may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

For example, acid addition salts are prepared from the free base using conventional methodology involving reaction of an active agent in the form of a free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, and inorganic acids, e.g., hydrochloric acid, hydrobromic acid, sulfuric acid, nitric acid, phosphoric acid, and the like. An acid addition salt may be reconverted to the free base by treatment with a suitable base. Examples of acid addition salts of active agents useful in conjunction with the methods and compositions of the invention include raloxifene hydrochloride, arzoxifene hydrochloride, cis-1-[(4-(1-imidazolylmethyl)cyclohexyl)methyl]imidazole succinic acid (CGS 14796C), bis(p-cyanophenyl)imidazo-1-yl methane hemisuccinate (CGS 18320B), and the like. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Examples of basic addition salts of active agents useful in conjunction with the methods and compositions of the invention include diclofenac sodium, etodolac sodium, naproxen sodium, meclofenamate sodium, oxaprozin potassium, and diclofenac potassium.

Preparation of esters involves transformation of a carboxylic acid group via a conventional esterification reaction involving nucleophilic attack of an RO— moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Examples of esters useful herein include 4-tert-butylcyclohexyl-4-pyridylacetate and Amides may be prepared from esters, using suitable amine reactants, or they may be prepared from an anhydride or an acid chloride by reaction with ammonia or a lower alkyl amine. Prodrugs, active metabolites, etc. may also be prepared using techniques known to those skilled in the art or described in the pertinent literature. Prodrugs are typically prepared by covalent attachment of a moiety that results in a compound that is therapeutically inactive until modified by an individual's metabolic system. Examples of conjugates useful herein are fulvestrant derivatives in which conjugates with glucose or cellobiose are prepared at the 3-hydroxyl and/or 17-hydroxyl moieties. See Thompson et al. (2003) Tet. Lett. 45 (6):1207-10.

Other derivatives and analogs of the active agents may be prepared using standard techniques known to those skilled in the art of synthetic organic chemistry, or may be deduced by reference to the pertinent literature. For those active agents that are chiral in nature and can thus be in enantiomerically pure form or in a racemic mixture, the active agent may be incorporated into the present compositions and delivery systems either as the racemate or in enantiomerically pure form.

### Dosage

Generally, a therapeutically effective dosage can be estimated initially in cell culture assays, e.g., using endometrial cells or smooth muscle cells from fibroids of neoplastic cells, or in animal models, using mice, rabbits, primates, etc. The animal model may also be used to determine the appropriate concentration range for the compositions herein. Generally, the amount of any one of the active agents herein administered in a single dose will be in the range of: about 0.01 mg to about 50 mg, preferably about 0.25 mg to about 25 mg, of the aromatase inhibitor, with preferred doses for specific aromatase inhibitors set forth below; about 0.5 mg to about 7.5 g, preferably about 10 mg to about 1500 mg, of the antiinflammatory agent, with preferred doses for specific antiinflammatory agents set forth below; and about 0.25 mg to about 500 mg, preferably about 0.5 mg to about 250 mg, of the uterine-selective estrogen receptor antagonist, again with preferred doses for specific such agents set forth below. The dosage selected will depend, of course, on the specific active agent and its potency as well as on the indication, the severity of the condition, the age, weight, and general health of the individual, and other pertinent factors known to the prescribing physician.

With respect to typical aromatase inhibitors, the present invention contemplates administering about 0.01 mg/day to about 2 mg/day of anastrozole, preferably about 0.1 mg/day to about 1 mg/day, with a specific dose being about 1 mg/day; about 0.25 mg/day to about 50 mg/day of exemestane, preferably about 2.5 mg/day to about 25 mg/day, with a specific dose being about 25 mg/day; and about 0.025 mg/day to about 5 mg/day of letrozole, preferably about 0.25 mg/day to about 2.5 mg/day, with a specific dose being about 2.5 mg/day.

With regard to typical non-steroidal antiinflammatory drugs, the present invention contemplates administering about 2 mg/day to about 400 mg/day of aceclofenac, preferably about 20 mg/day to about 200 mg/day, with a specific dose of about 200 mg/day; about 20 mg/day to about 8,000 mg/day of aspirin, preferably about 200 mg/day to about 4,000 mg/day, with a specific dose in the range of about 2,000 mg/day to about 4,000 mg/day; about 0.5 mg/day to about 150 mg/day of dexketoprofen, preferably about 5 mg/day to about 75 mg/day, with a specific dose in the range of about 50 mg/day to about 75 mg/day; about 1 mg/day to about 400 mg/day of diclofenac, preferably about 10 mg/day to about 150 mg/day, with a specific dose in the range of about 100 mg/day to about 150 mg/day; about 10 mg/day to about 3,000 mg/day of diflunisal, preferably about 100 mg/day to about 1,500 mg/day, with a specific dose in the range of about 1,000 mg/day to about 1,500 mg/day; about 4 mg/day to about 2,000 mg/day of etodolac, preferably about 40 mg/day to about 1,000 mg/day, with a specific dose in the range of about 400 mg/day to about 1,000 mg/day; about 12 mg/day to about 4,800 mg/day of fenoprofen, preferably about 120 mg/day to about 2,400 mg/day, with a specific dose in the range of about 1,200 mg/day to about 2,400 mg/day; about 6 mg/day to about 1,200 mg/day of flufenamic acid/day, preferably about 60 mg/day to about 600 mg/day, with a specific dose of about 600 mg/day; about 2 mg/day to about 600 mg/day of flurbiprofen, preferably about 20 mg/day to about 300 mg/day, with a specific dose in the range of about 200 mg/day to about 300 mg/day; about 12 mg/day to about 6,400 mg/day of ibuprofen, preferably about 120 mg/day to about 3,200 mg/day, with a specific dose in the range of about 1,200 mg/day to about 3,200 mg/day; about 1 mg/day to about 400 mg/day of indomethacin, preferably about 10 mg/day to about 200 mg/day, with a specific dose in the range of about 100 mg/day to about 200 mg/day; about 2 mg/day to about 600 mg/day of ketoprofen, preferably about 20 mg/day to about 300 mg/day, with a specific dose in the range of about 200 mg/day to about 300 mg/day; about 0.2 mg/day to about 80 mg/day of ketorolac, preferably about 2 mg/day to about 40 mg/day, with a specific dose in the range of about 20 mg/day to about 40 mg/day; about 0.08 mg/day to about 32 mg/day of lornoxicam, preferably about 0.8 mg/day to about 16 mg/day, with a specific dose in the range of about 8 mg/day to about 16 mg/day; about 2 mg/day to about 600 mg/day of meclofenamic acid, preferably about 20 mg/day to about 300 mg/day, with a specific dose in the range of about 200 mg/day to about 300 mg/day; about 5 mg/day to about 2,000 mg/day of mefenamic acid, preferably about 50 mg/day to about 1,000 mg/day, with a specific dose in the range of about 500 mg/day to about 1,000 mg/day; about 0.075 mg/day to about 30 mg/day of meloxicam, preferably about 0.75 mg/day to about 15 mg/day, with a specific dose in the range of about 7.5 mg/day to about 15 mg/day; about 10 mg/day to about 4,000 mg/day of nabumetone, preferably about 100 mg/day to about 2,000 mg/day, with a specific dose in the range of about 1,000 mg/day to about 2,000 mg/day; about 5 mg/day to about 3,000 mg/day of naproxen, preferably about 25 mg/day to about 1,500 mg/day, with a specific dose in the range of about 50 mg/day to about 1,000 mg/day; about 12 mg/day to about 2,400 mg/day of oxaprozin, preferably about 120 mg/day to about 1,200 mg/day, with a specific dose of about 1,200 mg/day; about 0.2 mg/day to about 40 mg/day of piroxicam, preferably about 2 mg/day to about 20 mg/day, with a specific dose in the range of about 5 mg/day to about 10 mg/day; about 3 mg/day to about 800 mg/day of sulindac, preferably about 30 mg/day to about 400 mg/day, with a specific dose in the range of about 300 mg/day to about 400 mg/day; about 6 mg/day to about 1,200 mg/day of tiaprofenic acid, preferably about 60 mg/day to about 600 mg/day, with a specific dose of about 600 mg/day; and about 12 mg/day to about 3,600 mg/day of tolmetin, preferably about 120 mg/day to about 1,800 mg/day, with a specific dose in the range of about 1,200 mg/day to about 1,800 mg/day.

With regard to typical uterine-selective estrogen receptor antagonists, the present invention contemplates administering about 0.25 mg/day to about 500 mg/month of fulvestrant, preferably about 25 mg/day to about 250 mg/month, with a specific dose being about 125 mg/month; about 0.12 mg/day to about 240 mg/week of ormeloxifene, preferably about 1.2 mg/day to about 120 mg/week, with a specific dose being about 60 mg/week; and about 0.6 mg/day to about 120 mg/day of raloxifene, preferably about 6 mg/day to about 60 mg/day, with a specific dose being about 60 mg/day.

Although broad ranges for each ingredient have been given above, it is anticipated that, by using a combination of ingredients in the compositions of the present invention, they would act synergistically, thus allowing lower doses of each to be used while retaining clinical efficacy with a better side effect profile.

The compositions of the present invention may be administered daily, weekly, monthly, or even annually, depending in part upon the need of each particular individual and the type of composition or delivery system utilized. Preferably, the compositions of the present invention are administered daily, once a week, once every two weeks, or once a month. This combination therapy will reduce estrogen production, block estrogen action, and suppress inflammation locally and synergistically, resulting in starvation of the estrogen-dependent disease tissues, relief of related symptoms, and retardation of disease progression, achieving symptom relief and disease modifying therapeutic efficacy. Once those beneficial results have been achieved and verified, the individual may be placed on a maintenance regimen where the administration of the combination therapy of the present invention will be administered periodically as needed to maintain those beneficial results without necessarily having the same administration regimen as when the combination therapy was initially undertaken. This, as would be expected, may vary from individual to individual.

All of the specifically named active agents herein are known; some are commercially available, and for those that are not, references may be had to the pertinent texts, literature, and patents that describe methods for the chemical synthesis thereof.

Compositions and delivery systems for intravaginal administration according to the present method include, without limitation, ointments, creams, lotions, emulsions, gels, solutions, suspensions, pastes, foams, films, suppositories, vaginal rings (see, e.g., U.S. Pat. No. 5,188,835 to Lindskog et al.), sponges, tampons, osmotic pump systems, intrauterine devices (IUDs), and intrauterine systems (IUS). Each of the aforementioned delivery systems will deliver the components of the compositions described herein to the site or sites (e.g., the uterus) where they are absorbed locally to achieve the beneficial effects of this invention.

The pharmaceutical compositions contain one or more pharmaceutically acceptable carriers and/or excipients suitable for incorporation into a formulation or delivery system for intravaginal administration, and selected according to the particular type of formulation, i.e., gel, ointment, vaginal suppository, or the like. In general, these auxiliary agents are physiologically acceptable and may be naturally occurring or may be of synthetic origin. Ideally, the carriers and/or excipients will be gradually broken down into innocuous substances in the body, or are of a nature that allows them to be secreted by the vagina and washed cleanly from the skin. In either case, they do not foul or clog the pores in skin or mucous membranes, leave any unacceptable residues, or cause other adverse effects. Such additives include, by way of example, liquid carriers (e.g., water or saline), preservatives, thickening agents, lubricating agents, permeation enhancers, emulsifying agents, pH buffering agents, disintegrating agents, binders, coloring agents, viscosity controlling agents, and the like. Mucoadhesive agents such as hydroxypropyl methylcellulose (HPMC) for facilitating prolonged contact with the vaginal wall are also exemplary excipients.

Representative preservatives for use in the present compositions include butylated hydroxyanisole, butylated hydroxytoluene, chlorobutanol, ethylenediamine, ethylparaben, methylparaben, monothioglycerol, phenol, phenylethyl alcohol, propylparaben, sodium benzoate, sodium formaldehyde sulfoxylate, sodium metabisulfite, sorbic acid, sulfur dioxide, maleic acid, and propyl gallate.

Thickening agents include, by way of example, carrageenans (iota, kappa, or lambda; see Published U.S. Patent Application No. 2005/0239742 A1 to Place et al.), acacia, agar, alginate, gum tragacanth, xanthan gum, collagen, carboxypolymethylene, polyvinylpyrrolidone, polyacrylamide, and cellulosic polymers, particularly the more hydrophilic cellulose derivatives.

Exemplary lubricating agents include glycerin (also called glycerine, glycerol, 1,2,3-propanetriol, and trihydroxypropane) and certain types of polyethylene glycol (PEG), such as PEG 200 or PEG 400. Various other polymers (such as polypropylene glycol, polyisobutene, and polyethylene oxide) and certain naturally occurring compounds (such as behenic acid, derived from various types of seeds and animal fats) and their derivatives (such as behenyl alcohol) may be used as well. Sugars and sugar alcohols such as sorbitol, mannitol, and lactose, and some silicon compounds such as polydimethylsiloxane, may also be used. Preferred supplemental lubricating agents include glycerin, propylene glycol, polyethylene glycol, and polypropylene glycol, due to their demonstrated biocompatibility, ease of synthesis, and widespread commercial availability.

To ensure that the composition is retained in the vaginal cavity for a suitable time period and not easily washed away, a bioadhesive component such as a crosslinking agent may also be incorporated into the composition.

The compositions may also include a chemical compound to enhance permeation of the active agent through the mucosal tissue, i.e., a “permeation enhancer.” Suitable permeation enhancers include those generally useful in conjunction with topical, transdermal and/or transmucosal drug delivery. Examples of suitable permeation enhancers include the following: sulfoxides such as dimethylsulfoxide decylmethylsulfoxide; ethers such as diethylene glycol monoethyl ether and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); and amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine. Mixtures of two or more enhancers may also be used.

As noted above, the pharmaceutical compositions herein may be in the form of an ointment, cream, lotion, emulsion, gel, solution, paste, suspension, foam, film, vaginal suppository (or a “pessary”), liposomal formulation, etc. The composition may also be incorporated into a device-type delivery system as will be described infra.

Ointments are semisolid preparations that are typically based on petrolatum or other petroleum derivatives; as with other pharmaceutically acceptable carriers useful herein, the ointment base should be inert, stable, nonirritating, and nonsensitizing. Ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Pharmaceutical creams are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

Lotions are preparations that may be applied without friction, and are typically liquid or semiliquid preparations in which solid particles, including the active agent, are present in a water or alcohol base along with a suspending agent; lotions are typically emulsions of the oil-in-water type. In general, pharmaceutical emulsions are generally formed from a dispersed phase (e.g., a pharmacologically active agent), a dispersion medium, and an emulsifying agent.

The aforementioned pharmaceutical compositions for intravaginal administration are formed by dispersing the finely divided or dissolved active agent uniformly throughout the vehicle or base using conventional techniques, typically by a levigating the agent with a small quantity of the base to form a concentrate, which is then diluted geometrically with further base. Alternatively, a mechanical mixer may be used. Creams, lotions and other emulsions are formed by way of a two-phase heat system, wherein oil-phase ingredients are combined under heat to provide a liquefied, uniform system. The aqueous-phase ingredients are separately combined using heat. The oil and aqueous phases are then added together with constant agitation and allowed to cool. At this point, concentrated agents may be added as a slurry. Volatile or aromatic materials can be added after the emulsion has sufficiently cooled. Preparation of such pharmaceutical formulations is within the general skill of the art.

The active agent can also be incorporated into a gel formulation using known techniques. Two-phase gel systems generally comprise a suspension or network of small, discrete particles interpenetrated by a liquid to provide a dispersed phase and a liquid phase. Single-phase gel systems are formed by distributing organic macromolecules uniformly throughout a liquid such that there are no apparent boundaries between the dispersed and liquid phases. Suitable gelling agents for use herein include synthetic macromolecules (e.g., carbomers, polyvinyl alcohols and polyoxyethylene-polyoxypropylene copolymers), gums such as tragacanth, as well as sodium alginate, gelatin, methylcellulose, sodium carboxymethylcellulose, methylhydroxyethyl cellulose and hydroxyethyl cellulose. In order to prepare a uniform gel, dispersing agents such as alcohol or glycerin can be added, or the gelling agent can be dispersed by trituration, mechanical mixing or stirring, or combinations thereof.

Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations.

Vaginal suppositories are typically manufactured with polyethylene glycol (PEG), polyethylene oxide and/or other low melting point or water-soluble polymers including fatty acid esters. Suppositories are intended to melt relatively rapidly following insertion into the vagina, but may be formulated so as to provide for sustained release of the active agents.

Typically, the present compositions contain each active agent in a concentration such that an effective amount of the agent is delivered with a single application of the composition; this amount is referred to as a “unit dose.” With ointments, creams, lotions, gels, etc., the concentration of each active agent in the composition should be sufficient to enable delivery of a unit dose by application of about 0.05 g to about 5.0 g of the composition, typically in the range of about 0.1 g to about 2.5 g of the composition, and more typically in the range of about 0.1 g to about 1.0 g of the composition. With vaginal suppositories, a typical suppository weight is in the range of about 0.1 g to 0.5 g, meaning that the concentration of each active agent therein should be such that a unit dose is provided in 0.1 g to 0.5 g of the suppository formulation.

Methods for manufacturing compositions for intravaginal administration are known in the art and described, for example, in Remington: The Science and Practice of Pharmacy, Nineteenth Ed. (Easton, Pa.: Mack Publishing Company, 1995). See also U.S. Patent Application No. 2005/0049231 A1 to Knox et al., cited supra, the disclosure of which is incorporated herein, including the cited patent documents.

The active agents herein may also be administered intravaginally using a “device-type” delivery system such as a vaginal ring (see, e.g., U.S. Pat. No. 5,188,835 to Lindskog et al.), sponge, tampon, osmotic pump system, intrauterine device (IUD), or intrauterine system (IUS), as known in the art. See also U.S. Pat. No. 6,086,999 to Harrison et al. It will be appreciated that depending on the system selected, the active agents may be provided as a coating (e.g., on an IUD or IUS), impregnated into or absorbed by the system (e.g., sponge, tampon), or applied to the system by any means that allows the active agents to attach to, bond with, or otherwise become physically associated therewith in a manner that allows for intravaginal administration and delivery to the vaginal mucosa.

Each of the compositions and delivery systems herein will deliver the components therein to the site or sites (e.g., the vagina or the uterus) where they are intended to be absorbed locally over a given time period, depending upon the nature of the active agents and other components as well as on the composition or delivery system chosen, to achieve the beneficial effects of the present invention. The delivery forms may be prepared for prompt or immediate release of the ingredients of the compositions of the present invention or for controlled or sustained release over a longer period of time. Those of ordinary skill in the art can readily adapt the compositions and methods described herein to provide sustained release of the active agents; reference may also be had to the pertinent texts and literature. For example, the compositions and delivery systems herein may be made so as to contain a hydrocarbon base (e.g. white petrolatum), mucoadhesive agents as alluded to earlier herein (e.g., HPMC or chitosan), a gel-forming or thickening agent, e.g. sodium alginate, tragacanth, gelatin, methylcellulose, sodium carboxymethylcellulose, carbomer, or the like.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees C. and pressure is at or near atmospheric pressure at sea level. All reagents were obtained commercially unless otherwise indicated.

### EXAMPLE 1

A bioadhesive, biodegradable vaginal suppository for the treatment of endometriosis and uterine fibroids in the human female is prepared containing about 0.2 mg of anastrozole and about 1,000 mg of naproxen. It is intended to be inserted into the upper part of the vagina by the patient herself, once every two days. The vaginal suppository will remain in contact with the epithelial surfaces of the vaginal mucosa for a minimum of 30 minutes, but preferably for 48 hours.

### EXAMPLE 2

A bioadhesive, biodegradable vaginal suppository is prepared as in Example 1 to release about 1.0 mg of anastrozole and about 2,000 mg of naproxen.

### EXAMPLE 3

A bioadhesive vaginal suppository is prepared as in Example 1 to release about 1 mg of anastrozole and about 3,000 mg of naproxen.

### EXAMPLE 4

An intra-uterine device (IUD) for the treatment of endometriosis, uterine fibroids and uterine cancer is prepared to release about 2.5 mg/day of letrozole and about 250 mg/month of fulvestrant. The IUD can be inserted into the uterus by a trained medical practitioner during the patient's office visit. The IUD has a drug cylinder that releases the letrozole and the fulvestrant inside the uterus. The drug cylinder is made of a biodegradable polymer soaked with appropriate amount of letrozole and fulvestrant. The drug release rate is determined by the rate of degradation of the polymer. The drug cylinder will be designed and formulated to last for about 90 days. It can be easily removed by a trained medical practitioner using a pair of forceps to take hold of the IUD's thread and gently retract, or by the woman herself.

### EXAMPLE 5

An IUD is prepared as in Example 4 to release about 1.25 mg/day of letrozole and 125 mg/month of fulvestrant.

### EXAMPLE 6

An IUD is prepared as in Example 4 to release about 0.625 mg/day of letrozole and 75 mg/month of fulvestrant.

### EXAMPLE 7

A bioadhesive vaginal ring for the treatment of endometriosis and uterine fibroids in the human female is prepared to release about 12 mg/week of ormeloxifene and about 5 mg/day of piroxicam. Each of the ingredients will be soaked into a biodegradable polymer that will be placed inside the porous rubber-like ring. The soft, flexible ring is inserted into the upper part of the vagina by a patient or a trained medical practitioner. The ring releases a consistent dose of ormeloxifene and piroxicam while in place over one week and then is replaced on a weekly basis as long as needed to treat the woman's condition.

### EXAMPLE 8

A vaginal ring is prepared as in Example 7 to release about 60 mg/week of ormeloxifene and about 10 mg/day of piroxicam.

### EXAMPLE 9

A vaginal ring is prepared as in Example 7 to release about 120 mg/week of ormeloxifene and about 20 mg/day of piroxicam.

### EXAMPLE 10

A vaginal ring for the treatment of endometriosis, uterine fibroids and uterine cancer in the human female is prepared as in Example 7 to release about 0.1 mg/day of anastrozole, 5 mg/day of piroxicam, and 12 mg/week of ormeloxifene. The ring releases the doses of anastrozole, ormeloxifene and piroxicam while in place.

### EXAMPLE 11

A vaginal ring is prepared as in Example 7 to release about 0.5 mg/day of anastrozole, 10 mg/day of piroxicam, and 60 mg/week of ormeloxifene.

### EXAMPLE 12

A vaginal ring is prepared as in Example 7 to release about 1 mg/day of anastrozole, about 20 mg/day of piroxicam, and about 120 mg/week of ormeloxifene.

### EXAMPLE 13

A bioadhesive, biodegradable vaginal pellet for the treatment of endometriosis and uterine fibroids in the human female is prepared containing about 25 mg of exemestane and about 60 mg of raloxifene. The pellet can be inserted into the upper part of the vagina by the patient herself once a day.

### EXAMPLE 14

A bioadhesive, biodegradable vaginal pellet is prepared as in Example 13, but contains about 12.5 mg of exemestane and about 30 mg of raloxifene.

### EXAMPLE 15

A bioadhesive, biodegradable vaginal pellet is prepared as in Example 13, but contains about 6.25 mg of exemestane and about 15 mg of raloxifene.

### EXAMPLE 16

A bioadhesive, biodegradable vaginal suppository for the treatment of endometriosis and uterine fibroids in the human female is prepared containing about 0.10 mg of anastrozole and about 100 mg of diclofenac. It is intended to be inserted into the upper part of the vagina by the patient herself daily. The vaginal suppository will remain in contact with the epithelial surfaces of the vaginal mucosa for a minimum of 30 minutes, but preferably for 24 hours.

### EXAMPLE 17

A bioadhesive, biodegradable vaginal suppository is prepared as in Example 16, but contains about 0.5 mg of anastrozole and about 100 mg of diclofenac.

### EXAMPLE 18

A bioadhesive, biodegradable vaginal suppository is prepared as in Example 16, but contains about 1 mg of anastrozole and about 150 mg of diclofenac.

### EXAMPLE 19

A bioadhesive, biodegradable vaginal tablet for the treatment of endometriosis and uterine fibroids in the human female is prepared containing about 60 mg of raloxifene and about 15 mg of meloxicam. It is intended to be inserted into the upper part of the vagina by the patient herself on a daily basis and will remain in contact with the epithelial surfaces of the vaginal mucosa until dissolved.

### EXAMPLE 20

A bioadhesive, biodegradable vaginal tablet is prepared as in Example 19, but contains about 30 mg of raloxifene and about 10 mg of meloxicam.

### EXAMPLE 21

A bioadhesive, biodegradable vaginal tablet is prepared as in Example 19, but contains about 15 mg of raloxifene and about 7.5 mg of meloxicam.

### EXAMPLE 22

An intrauterine system (IUS) for the treatment of endometriosis, uterine fibroids and uterine cancer in the human female is prepared to release about 25 mg/day of exemestane, 250 mg/month of fulvestrant and 1,500 mg/day of naproxen. The IUS is inserted into the uterus by a trained medical practitioner and continuously releases exemestane, fulvestrant and naproxen at sustained levels while in place. It is intended to be left in place for one month.

### EXAMPLE 23

An IUS is prepared as in Example 22 to release about 12.5 mg/day of exemestane, about 125 mg/month of fulvestrant and about 1,000 mg/day of naproxen.

### EXAMPLE 24

An IUS is prepared as in Example 22 to release about 6.25 mg/day of exemestane, about 62.5 mg/month of fulvestrant and about 500 mg/day of naproxen.

While various embodiments of the present invention have been described, it should be understood that various modifications and adaptations thereof will be apparent to one skilled in this art. Such modifications and adaptations are considered to be within the scope of the present invention, which is limited only by the scope of the following claims.

1. A method for treating a subject for an estrogen-dependent hyperproliferative uterine condition, comprising intravaginally administering to the subject a combination of at least two active agents selected from an aromatase inhibitor, an antiinflammatory agent, and a uterine-selective estrogen receptor antagonist.

2.-40. (canceled)
